(19) United States
(12) Patent Application Publication
(10) Pub. No.: US 2002/0103489 A1
(43) Pub. Date: Aug. 1, 2002

(54) Medical staple

(76) Inventor: Bon-Hie Ku, Busanjin-Gu Busan (KR)

Correspondence Address: BIRCH STEWART KOLASCH & BIRCH, PO BOX 747, FALLS CHURCH, VA, 22040-0747, US

(21) Appl. No.: 10/055,347

(22) Filed: Jan. 25, 2002

(30) Foreign Application Priority Data
Jan. 26, 2001 (KR) 2001-1876

(51) Int. Cl. A61B 17/56

(52) U.S. Cl. 606/75

(57) ABSTRACT

The present invention relates to a medical staple having the compressive and expandable leg member which is used for the orthopedic surgery, the surgery of an calcaneus, talus fracture in order to repair broken bones in a safe and effective way, more particularly it is directed to devise a new type of medical staple for the bone setting which is capable of adjusting the distance between staple's leg member so as to effectively control the compressional strength onto the contacting portion of broken bones depending on the clinical diagnosis after the surgery of bone setting.

### BACKGROUND OF THE INVENTION

1. Field of the Invention

The present invention relates to a medical staple having a compressible and expandable leg member which is used in orthopedic surgery, e.g., in the surgery of a calcaneus, talus fracture in order to repair broken bones in a safe and effective manner. More particularly, the present invention is directed to a new type of medical staple for bone setting which is capable of adjusting the distance between the staple's leg members so as to effectively control the compressional strength applied to the contacting portions of broken bones after bone setting surgery.

2. Description of Related Art

Most conventional medical staples in the prior art show an unitary shape having two parallel pins extending downwardly from the edges of a bridge member which defines a fixed distance between the parallel pins, as shown in FIG. 4. The conventional staples having an unitary shape of the leg member with a bridge member, have the following problems due to the preset distance of the parallel legs and no way for adjusting the distance between the leg members.

As a preliminary procedure for the medical operation involving bone setting, the surgical operation of drilling a pair of holes on opposite sides of the fractured or crooked bones in order to insert staple pins across the fracture requires a lot of skill and experience on the part of the doctor. Furthermore, an optimized vertical angle of the holes compatible with the shape of the staple's pins becomes a very important factor. Thus, a lot of carefulness and skillfulness for securing bone fractures using the preset-distance staples of the prior art is required.

Although the surgical operation in connection with providing an exact location and desirable angle of holes to be formed on the remediable bones can be successfully completed using the medical staples of the prior art, other problems are likely to occur, i.e., the contacting portions of the fractured bones do not tightly knit together due to the preset distance of leg members of the staple. Thus, since the prior art staples provide no flexible means for tightening or loosening the compression force to close any gaps in the contacting portion of the fracture, such gaps tend to increase since blood circulation is not effective across a cracked bone in which a crevice is formed.

In other words, when using the medical staples of the prior art, there is no typical embodiment or function for treating the mysterious death of bone around a crevice by effectively controlling the compression strength between broken bones in order to correct this necrosis phenomena. Therefore, the medical staples of the prior art, having fixed leg members aggravate the clinical state of a patient after bone surgery and bone setting and accordingly re-surgery or re-setting of the bone is frequently required.

### SUMMARY OF THE INVENTION

Accordingly, the medical staple of the present invention is directed towards improving upon the disadvantages of prior art which use medical staples have a fixed distance between the legs thereof.

The present invention provides a medical staple having a movable leg member and a fastening means for securing the movable leg along a horizontal bar, thereby adjusting and controlling the distance between leg members, either expansively or compressively.

In practice, a pair of apertured holes to be formed in broken or fractured bones requires the exact positioning thereof in order to achieve a tight and secure knit of adjacent bone with the staple's legs.

The present invention provides a staple with movable legs, thereby eliminating the strict requirements for precise location holes for matching with the staple's legs. This gives surgeons flexibility in drilling holes for setting bone.

Furthermore, the use of staples where the distance between leg members can be adjusted, enhances the bonding effect between broken bones, avoids the mysterious death which can occur in the bone around crevices between the break, and shortens the recovery time of the bond setting procedure by accelerating the blood circulation around the contacting portions of the bone.

Other objects and further scope of applicability of the present invention will become apparent from the detailed description given hereinafter. It should be understood, however, that the detailed description and specific examples, while indicating preferred embodiments of the invention, are given by way of illustration only, since various change and modification within the spirit and scope of the invention will become apparent to those skilled in the art from this detailed description.

### BRIEF DESCRIPTION OF THE DRAWINGS

The accompanying drawings, which are included to provide further understanding of the present invention and are incorporated in and constitute part of this specification, illustrate embodiments of the invention and together with the description serve to explain the principles of the invention.

FIG. 1 is a perspective view of a medical staple which shows the embodiment of the present invention;

FIG. 2 is a cross sectional view of the medical staple which shows a preliminary state before the joining of the medical staple and the remediable bones, under the embodiment of present invention;

FIG. 3 is a cross sectional view of the medical staple which shows the bonding state after the joining of the medical staple and the remediable bones together under the embodiment of the present invention; and

FIG. 4 is a cross sectional view of the medical staple of the prior art which has a preset distance between the pins of the staple.

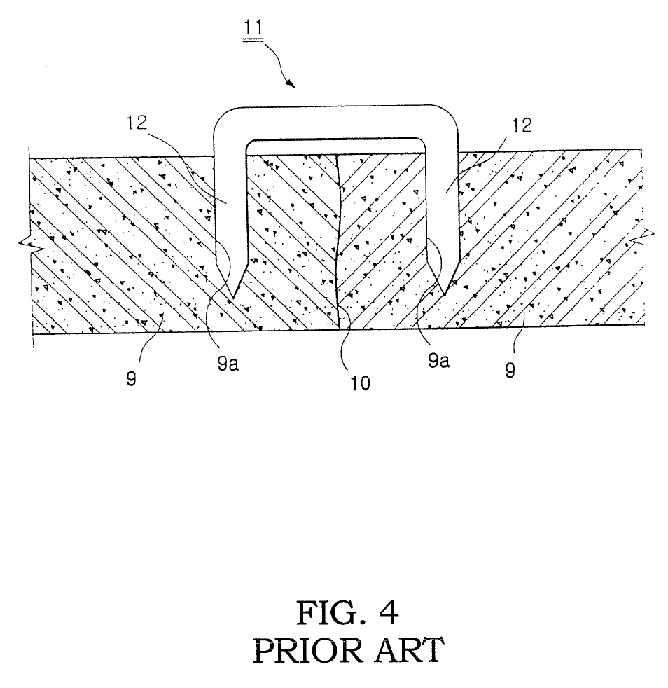

### DETAILED DESCRIPTION OF THE PREFERRED EMBODIMENTS

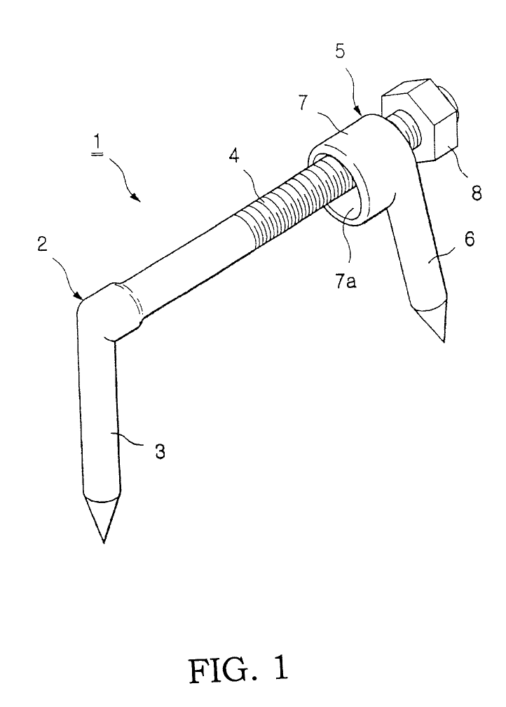

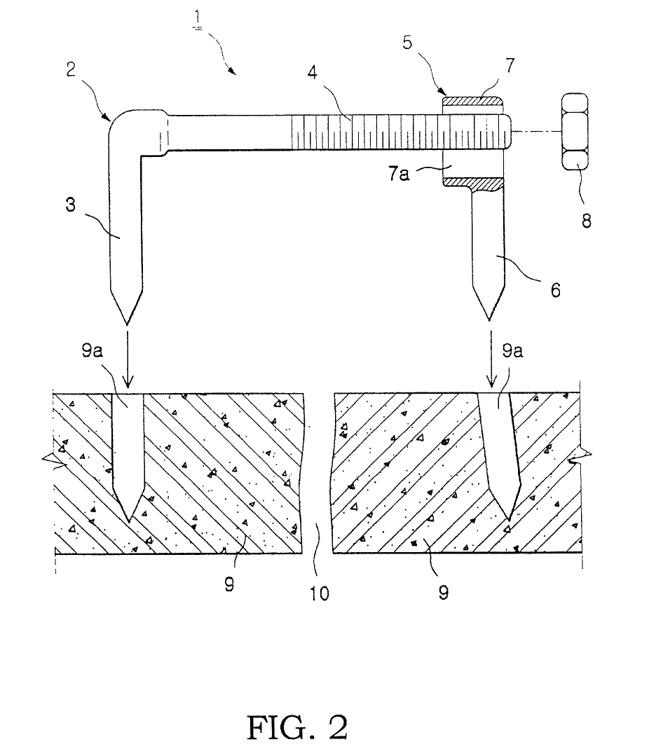

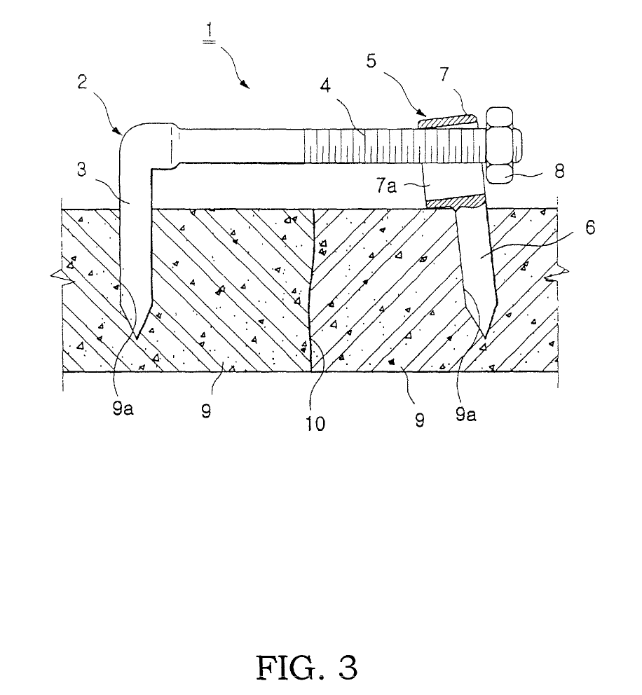

Referring now in detail to the drawings for the purpose of illustrating the preferred embodiments of the present invention, the medical staple 1 as shown in FIGS. 1, 2 and 3 includes a fastening means 8 and a pair of movable leg members 3, 6, made of a metal such as Titanium or a metal compound which is innocuous or inert to human body. The purpose of the medical staple is to secure the crevice portion 10 of the fractured bones 9 together by joining the components of the medical staple together.

More specifically, the medical staple 1 of the present invention comprises an L-square pin 2 having a screw portion 4 disposed along the horizontal part of the L-square pin 2 and an insertable leg member 3 positioned to be substantially perpendicular to the horizontal portion of the L-square pin 2. To join with the L-square pin 2, a separable leg member 5 shaped as an insertable pin 6 with a head tube 7 having a through hole 7a provided for adjusting the distance using the screw portion 4 of the L-square pin (2).

For the secure combinational joining for the L-square pin 2 and the separable leg member 5 as mentioned above, a fastening means 8, joinable to the screw portion 4 of the horizontal portion of L-square pin 2 is used to secure the separable leg member 5 to the L-square pin 2 resulting in a substantially parallel and spaced-apart relationship for the combination into the apertured holes 9a of the fractured bones.

Practically, the medical staple according to the present invention is devised for use in surgical operations for bone setting and is applicable to the same methods and procedures involving conventional staples.

A pair of holes 9a for accommodating the insertable pin 6 of the separable leg member 5 and the insertable leg member 3 of the L-square pin 2 are necessary to be formed in a precise way in the vertical and/or horizontal direction of the targeted bones. But according to the present embodiment, the perpendicularities for making holes 9a on the fractured bones and the exact location of holes compatible with the distance of the leg member would not confine the surgeon's operation for making the holes on the patient's bones.

Although the relative location and the vertical angle of the holes in the fractured bones may not be formed in an exact way, the fastening means 8 which is joinable with the screw portion 4 of the L-square pin 2 can effectively compensate for any differences in the relative distances and variances in perpendicularity of the holes, thereby providing a device with adjustable flexibility.

Since the through-hole 7a of the head tube 7 is shaped larger than a diameter of the screw portion 4 and the through hole 7a is covered by a cylindrical shell, the inclination of the inserting angle of the medical staple for combining with holes 9a which are not precisely formed, is more flexible when compared with the fixed type medical staple of the prior art.

Furthermore, the compression force provided by the medical staple secured with the fastening means 8 provides a flexible way for compensating for correcting the phenomena of death around the crevice of fractured bones, resulting in the shortening of the duration of the medical treatment.

As mentioned above, the medical staple 1 separable into component parts consisting of the L-square pin 2, a separable leg member 5 and a fastening means 8 can be manufactured using the advantages of a relatively simple, recycling and substitutable structure resulting a production cost reduction. Furthermore, a medical staple having an expandable and compressible function as defined by the present invention provides a new type of surgical device for bone setting which leads to a simple, safe and effective method for medical recovery in the bonding of bones.

The invention being described, it will be obvious that the same may be varied in many ways, and such variations are not to be regarded as a departure from the spirit and scope of the invention, but rather are intended to be included within the scope of the following claims.

What is claimed is:

1. A medical staple for surgical operations which comprises:
a substantially L-shaped first leg member having a screw portion 4 and an insertable end portion;
a separate second leg member having an aperture at one end portion and an insertable end portion at the other end thereof; and
a fastener adapted to join with the screw portion of the first leg member, wherein the aperture of the second leg member is slidable onto the screw portion of the first leg member where it is secured by the fastener, whereby the distances between the insertable leg portions of the first and second leg members can be varied.

2. The medical staple of claim 1, wherein the aperture of the second leg member has a larger dimension compared with the screw portion 4 of the first leg member.

3. The medical staple of claim 1, wherein the fastener is a nut which is adapted for screw engagement with the screw portion.
